(19) United States
(12) Patent Application Publication
(10) Pub. No.: US 2021/0259783 A1
(43) Pub. Date: Aug. 26, 2021

(54) Systems and Methods Related to Registration for Image Guided Surgery

(71) Applicant: INTUITIVE SURGICAL OPERATIONS, INC., SUNNYVALE, CA (US)

(72) Inventors: Teresa G. Gadda, Palo Alto, CA (US); Troy K Adebar, Mountain View, CA (US); Vincent Duindam, San Francisco, CA (US); Timothy D. Soper, San Jose, CA (US)

(21) Appl. No.: 17/253,743

(22) PCT Filed: Jun. 12, 2019
(86) PCT No.: PCT/US2019/036723

§ 371 Date: Dec. 18, 2020

Related U.S. Application Data
(60) Provisional application No. 62/686,854, filed on Jun. 19, 2018.

(51) Int. Cl. A61B 34/20; A61B 34/10

(52) U.S. Cl. CPC A61B 34/20; A61B 34/10; A61B 2034/2065; A61B 2034/2068; A61B 2034/2059; A61B 2034/105

(57) ABSTRACT

A system includes one or more processors configured to read instructions to cause the system to perform operations including accessing a set of model points of a model of an anatomic structure of a patient. The model points is associated with a model space. The operations further include collecting a set of measured points of the anatomic structure of the patient, the measured points being associated with a patient space, determining a set of matches between the set of model points and the set of measured points, determining a first plurality of weights for the set of matches, and registering the set of model points to the set of measured points based on the first plurality of weights to generate a first registration.

### CROSS-REFERENCE TO RELATED APPLICATIONS

This application claims the benefit of U.S. Provisional Application 62/686,854 filed Jun. 19, 2018, which is incorporated by reference herein in its entirety.

### FIELD

The present disclosure is directed to systems and methods for conducting an image-guided procedure, and more particularly to systems and methods for using registered real-time images and prior-time anatomic images during an image-guided procedure.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments (including surgical, diagnostic, therapeutic, or biopsy instruments) to reach a target tissue location. To assist with reaching the target tissue location, the location and movement of the medical instruments may be correlated with pre-operative or intra-operative images of the patient anatomy. With the image-guided instruments correlated to the images, the instruments may navigate natural or surgically created passageways in anatomic systems such as the lungs, the colon, the intestines, the kidneys, the heart, the circulatory system, or the like. Usually, such a correlation is determined based on a rigid match between the location and movement of the image-guided instruments and the pre-operative or intra-operative images of the patient anatomy. However, such a rigid match may affect the quality of the correlation, and thereby affect the quality of the image-guided procedure.

Accordingly, it would be advantageous to provide improved registration for performing image-guided procedures.

### SUMMARY

The embodiments of the invention are best summarized by the claims that follow the description.

In one illustrative embodiment, a method is performed by a computing system. The method includes accessing a set of model points of a model of an anatomic structure of a patient, the model points being associated with a model space and collecting a set of measured points of the anatomic structure of the patient, the measured points being associated with a patient space. The method further includes determining a set of matches between the set of model points and the set of measured points, determining a first plurality of weights for the set of matches, and registering the set of model points to the set of measured points based on the first plurality of weights to generate a first registration.

In another illustrative embodiment, a method is performed by a computing system. The method includes accessing a set of model points of a model of an anatomic structure of a patient, the model points being associated with a model space and collecting a set of measured points of the anatomic structure of the patient, the measured points being associated with a patient space. The method further includes determining a first plurality of weights for the set of model points respectively based on a target anatomic location and registering the set of model points to the set of measured points based on the first plurality of weights to generate a registration.

In yet another illustrative embodiment, a method is performed by a computing system. The method includes accessing a set of model points of a model of an anatomic structure of a patient, the model points being associated with a model space, and collecting a set of measured points of the anatomic structure of the patient, the measured points being associated with a patient space. The method further includes determining a first plurality of weights for the set of measured points respectively; and registering the set of model points to the set of measured points based on the first plurality of weights to generate a registration.

In yet another illustrative embodiment, a method is performed by a computing system. The method includes accessing a set of model points of a model of an anatomic structure of a patient, the model points being associated with a model space. The method further includes collecting a set of measured points of the anatomic structure of the patient, the measured points being associated with a patient space. The method further includes registering the set of model points with the set of measured points to generate a first registration, dividing the anatomic structure into a plurality of anatomic areas; generating a plurality of area registrations for the plurality of anatomic areas respectively based on the first registration; and generating a second registration for translating the model space to the patient space using the plurality of area registrations.

In yet another illustrative embodiment, a method is performed by a computing system. The method includes accessing a set of model points of a model of an anatomic structure of a patient, the model points being associated with a model space. The method further includes collecting a set of measured points of the anatomic structure of the patient, the measured points being associated with a patient space. The method further includes registering the set of model points with the set of measured points to generate a first registration; providing a patient anatomic image from a distal end location of a medical instrument; and determining a mismatch between the patient anatomic image and a first visual representation of the model from a first navigation path location, the first navigation path location being determined based on the distal end location and the first registration. The method further includes providing a second visual representation of the model from a second navigation path location different from the first navigation path location; receiving a match indication that the patient anatomic image matches the second visual representation of the model; and generating a second registration for translating the model space to the patient space based on the distal end location and the second navigation path location.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a teleoperated medical system according to some embodiments.

FIG. 2A is a simplified diagram of a medical instrument system according to some embodiments.

FIG. 2B is a simplified diagram of a medical instrument with an extended medical tool according to some embodiments.

FIGS. 3A and 3B are simplified diagrams of side views of a patient coordinate space including a medical instrument mounted on an insertion assembly according to some embodiments.

FIGS. 4A, 4B, 4C, and 4D illustrate the distal end of the medical instrument system of FIGS. 2, 3A, 3B, during insertion within a human lung according to some embodiments.

FIG. 5 is a flowchart illustrating a method of an image-guided surgical procedure or a portion thereof according to some embodiments.

FIGS. 6A, 6B, and 6C illustrate steps in segmentation processes that generate a model of human lungs of a patient for registration according to some embodiments.

FIG. 7 is a flow chart providing a method for updating the registration of the anatomic model to the patient anatomy according to some embodiments.

FIGS. 8A and 8B illustrate the distal end of the medical instrument system during insertion within a human lung according to some embodiments.

FIG. 9 is a flow chart providing a method for updating the registration of the anatomic model to the patient anatomy according to some embodiments.

FIG. 10 illustrates a model of human lungs of a patient for registration according to some embodiments.

FIG. 11 is a flow chart providing a method for updating the registration of the anatomic model to the patient anatomy according to some embodiments.

FIGS. 12 and 13 illustrate a display stage of a re-registration technique according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

In some instances well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

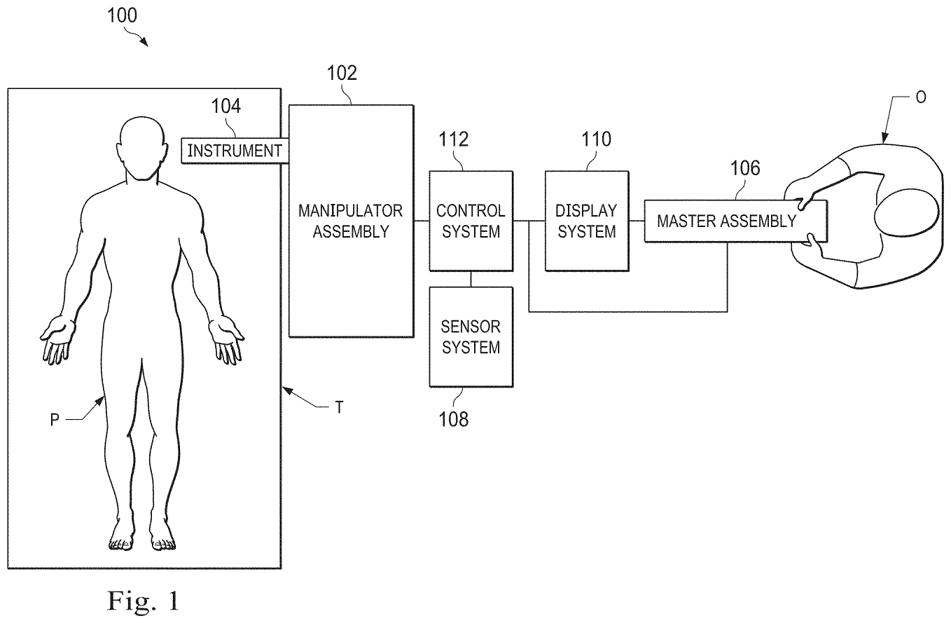

FIG. 1 is a simplified diagram of a teleoperated medical system 100 according to some embodiments. In some embodiments, teleoperated medical system 100 may be suitable for use in, for example, surgical, diagnostic, therapeutic, or biopsy procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic or teleoperational systems.

As shown in FIG. 1, medical system 100 generally includes a manipulator assembly 102 for operating a medical instrument 104 in performing various procedures on a patient P. The manipulator assembly 102 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. Manipulator assembly 102 is mounted to or near an operating table T. A master assembly 106 allows an operator (e.g., a surgeon, a clinician, or a physician as illustrated in FIG. 1) O to view the interventional site and to control manipulator assembly 102.

Master assembly 106 may be located at an operator console which is usually located in the same room as operating table T, such as at the side of a surgical table on which patient P is located. However, it should be understood that operator O can be located in a different room or a completely different building from patient P. Master assembly 106 generally includes one or more control devices for controlling manipulator assembly 102. The control devices may include any number of a variety of input devices, such as joysticks, trackballs, data gloves, trigger-guns, hand-operated controllers, voice recognition devices, body motion or presence sensors, and/or the like. To provide operator O a strong sense of directly controlling instruments 104 the control devices may be provided with the same degrees of freedom as the associated medical instrument 104. In this manner, the control devices provide operator O with telepresence or the perception that the control devices are integral with medical instruments 104.

In some embodiments, the control devices may have more or fewer degrees of freedom than the associated medical instrument 104 and still provide operator O with telepresence. In some embodiments, the control devices may optionally be manual input devices which move with six degrees of freedom, and which may also include an actuatable handle for actuating instruments (for example, for closing grasping jaws, applying an electrical potential to an electrode, delivering a medicinal treatment, and/or the like).

Manipulator assembly 102 supports medical instrument 104 and may include a kinematic structure of one or more non-servo controlled links (e.g., one or more links that may be manually positioned and locked in place, generally referred to as a set-up structure), and/or one or more servo controlled links (e.g. one more links that may be controlled in response to commands from the control system), and a manipulator. Manipulator assembly 102 may optionally include a plurality of actuators or motors that drive inputs on medical instrument 104 in response to commands from the control system (e.g., a control system 112). The actuators may optionally include drive systems that when coupled to medical instrument 104 may advance medical instrument 104 into a naturally or surgically created anatomic orifice. Other drive systems may move the distal end of medical instrument 104 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument 104 for grasping tissue in the jaws of a biopsy device and/or the like. Actuator position sensors such as resolvers, encoders, potentiometers, and other mechanisms may provide sensor data to medical system 100 describing the rotation and orientation of the motor shafts. This position sensor data may be used to determine motion of the objects manipulated by the actuators.

Teleoperated medical system 100 may include a sensor system 108 with one or more sub-systems for receiving information about the instruments of manipulator assembly 102. Such sub-systems may include a position/location sensor system (e.g., an electromagnetic (EM) sensor system); a shape sensor system for determining the position, orientation, speed, velocity, pose, and/or shape of a distal end and/or of one or more segments along a flexible body that may make up medical instrument 104; and/or a visualization system for capturing images from the distal end of medical instrument 104.

Teleoperated medical system 100 also includes a display system 110 for displaying an image or representation of the surgical site and medical instrument 104 generated by sub-systems of sensor system 108. Display system 110 and master assembly 106 may be oriented so operator O can control medical instrument 104 and master assembly 106 with the perception of telepresence.

In some embodiments, medical instrument 104 may have a visualization system (discussed in more detail below), which may include a viewing scope assembly that records a concurrent or real-time image of a surgical site and provides the image to the operator or operator O through one or more displays of medical system 100, such as one or more displays of display system 110. The concurrent image may be, for example, a two or three dimensional image captured by an endoscope positioned within the surgical site. In some embodiments, the visualization system includes endoscopic components that may be integrally or removably coupled to medical instrument 104. However in some embodiments, a separate endoscope, attached to a separate manipulator assembly may be used with medical instrument 104 to image the surgical site. The visualization system may be implemented as hardware, firmware, software or a combination thereof which interact with or are otherwise executed by one or more computer processors, which may include the processors of a control system 112.

Display system 110 may also display an image of the surgical site and medical instruments captured by the visualization system. In some examples, teleoperated medical system 100 may configure medical instrument 104 and controls of master assembly 106 such that the relative positions of the medical instruments are similar to the relative positions of the eyes and hands of operator O. In this manner operator O can manipulate medical instrument 104 and the hand control as if viewing the workspace in substantially true presence. By true presence, it is meant that the presentation of an image is a true perspective image simulating the viewpoint of a physician that is physically manipulating medical instrument 104.

In some examples, display system 110 may present images of a surgical site recorded pre-operatively or intra-operatively using image data from imaging technology such as, computed tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The pre-operative or intra-operative image data may be presented as two-dimensional, three-dimensional, or four-dimensional (including e.g., time based or velocity based information) images and/or as images from models created from the pre-operative or intra-operative image data sets.

In some embodiments, often for purposes of imaged guided surgical procedures, display system 110 may display a virtual navigational image in which the actual location of medical instrument 104 is registered (i.e., dynamically referenced) with the preoperative or concurrent images/model. This may be done to present the operator O with a virtual image of the internal surgical site from a viewpoint of medical instrument 104. In some examples, the viewpoint may be from a tip of medical instrument 104. An image of the tip of medical instrument 104 and/or other graphical or alphanumeric indicators may be superimposed on the virtual image to assist operator O controlling medical instrument 104. In some examples, medical instrument 104 may not be visible in the virtual image.

In some embodiments, display system 110 may display a virtual navigational image in which the actual location of medical instrument 104 is registered with preoperative or concurrent images to present the operator O with a virtual image of medical instrument 104 within the surgical site from an external viewpoint. An image of a portion of medical instrument 104 or other graphical or alphanumeric indicators may be superimposed on the virtual image to assist operator O in the control of medical instrument 104. As described herein, visual representations of data points may be rendered to display system 110. For example, measured data points, moved data points, registered data points, and other data points described herein may be displayed on display system 110 in a visual representation. The data points may be visually represented in a user interface by a plurality of points or dots on display system 110 or as a rendered model, such as a mesh or wire model created based on the set of data points. In some examples, the data points may be color coded according to the data they represent. In some embodiments, a visual representation may be refreshed in display system 110 after each processing operation has been implemented to alter data points.

Teleoperated medical system 100 may also include control system 112. Control system 112 includes at least one memory and at least one computer processor (not shown) for effecting control between medical instrument 104, master assembly 106, sensor system 108, and display system 110. Control system 112 also includes programmed instructions (e.g., a non-transitory machine-readable medium storing the instructions) to implement some or all of the methods described in accordance with aspects disclosed herein, including instructions for providing information to display system 110. While control system 112 is shown as a single block in the simplified schematic of FIG. 1, the system may include two or more data processing circuits with one portion of the processing optionally being performed on or adjacent to manipulator assembly 102, another portion of the processing being performed at master assembly 106, and/or the like. The processors of control system 112 may execute instructions comprising instruction corresponding to processes disclosed herein and described in more detail below. Any of a wide variety of centralized or distributed data processing architectures may be employed. Similarly, the programmed instructions may be implemented as a number of separate programs or subroutines, or they may be integrated into a number of other aspects of the teleoperational systems described herein. In one embodiment, control system 112 supports wireless communication protocols such as Bluetooth, IrDA, HomeRF, IEEE 802.11, DECT, and Wireless Telemetry.

In some embodiments, control system 112 may receive force and/or torque feedback from medical instrument 104. Responsive to the feedback, control system 112 may transmit signals to master assembly 106. In some examples, control system 112 may transmit signals instructing one or more actuators of manipulator assembly 102 to move medical instrument 104. Medical instrument 104 may extend into an internal surgical site within the body of patient P via openings in the body of patient P. Any suitable conventional and/or specialized actuators may be used. In some examples, the one or more actuators may be separate from, or integrated with, manipulator assembly 102. In some embodiments, the one or more actuators and manipulator assembly 102 are provided as part of a teleoperational cart positioned adjacent to patient P and operating table T.

Control system 112 may optionally further include a virtual visualization system to provide navigation assistance to operator O when controlling medical instrument 104 during an image-guided surgical procedure. Virtual navigation using the virtual visualization system may be based upon reference to an acquired preoperative or intraoperative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. Software, which may be used in combination with manual inputs, is used to convert the recorded images into segmented two dimensional or three dimensional composite representation of a partial or an entire anatomic organ or anatomic region. An image data set is associated with the composite representation. The composite representation and the image data set describe the various locations and shapes of the passageways and their connectivity. The images used to generate the composite representation may be recorded preoperatively or intra-operatively during a clinical procedure. In some embodiments, a virtual visualization system may use standard representations (i.e., not patient specific) or hybrids of a standard representation and patient specific data. The composite representation and any virtual images generated by the composite representation may represent the static posture of a deformable anatomic region during one or more phases of motion (e.g., during an inspiration/expiration cycle of a lung).

During a virtual navigation procedure, sensor system 108 may be used to compute an approximate location of medical instrument 104 with respect to the anatomy of patient P. The location can be used to produce both macro-level (external) tracking images of the anatomy of patient P and virtual internal images of the anatomy of patient P. The system may implement one or more electromagnetic (EM) sensor, fiber optic sensors, and/or other sensors to register and display a medical implement together with preoperatively recorded surgical images, such as those from a virtual visualization system. For example, PCT Publication WO 2016/191298 (published Dec. 1, 2016) (disclosing “Systems and Methods of Registration for Image Guided Surgery”), which is incorporated by reference herein in its entirety, discloses such one system. Teleoperated medical system 100 may further include optional operations and support systems (not shown) such as illumination systems, steering control systems, irrigation systems, and/or suction systems. In some embodiments, teleoperated medical system 100 may include more than one manipulator assembly and/or more than one master assembly. The exact number of teleoperational manipulator assemblies will depend on the surgical procedure and the space constraints within the operating room, among other factors. Master assembly 106 may be collocated or they may be positioned in separate locations. Multiple master assemblies allow more than one operator to control one or more teleoperational manipulator assemblies in various combinations.

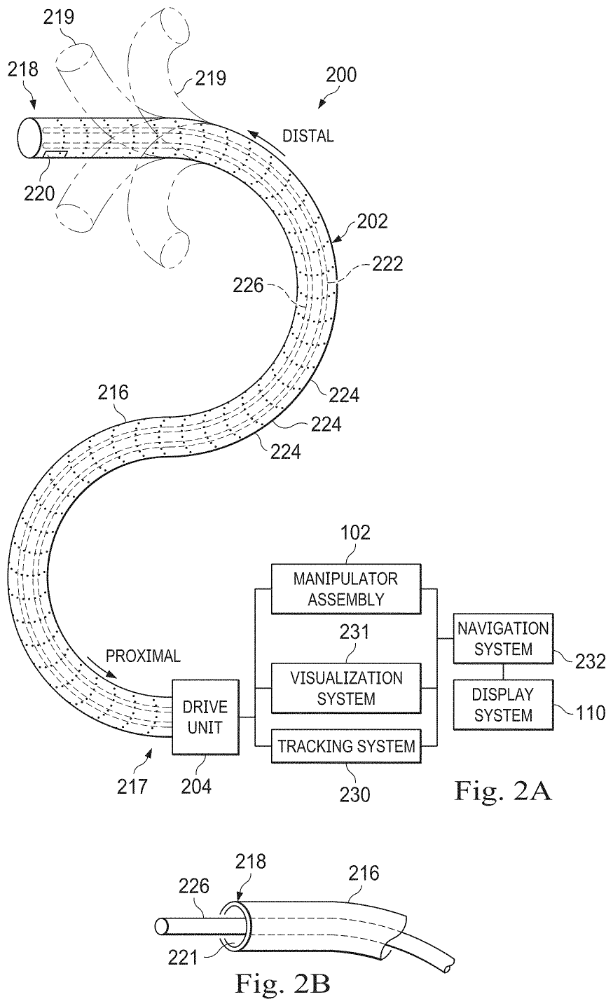

FIG. 2A is a simplified diagram of a medical instrument system 200 according to some embodiments. In some embodiments, medical instrument system 200 may be used as medical instrument 104 in an image-guided medical procedure performed with teleoperated medical system 100. In some examples, medical instrument system 200 may be used for non-teleoperational exploratory procedures or in procedures involving traditional manually operated medical instruments, such as endoscopy. Optionally medical instrument system 200 may be used to gather (i.e., measure) a set of data points corresponding to locations within anatomic passageways of a patient, such as patient P.

Medical instrument system 200 includes elongate device 202, such as a flexible catheter, coupled to a drive unit 204. Elongate device 202 includes a flexible body 216 having proximal end 217 and distal end or tip portion 218. In some embodiments, flexible body 216 has an approximately 3 mm outer diameter. Other flexible body outer diameters may be larger or smaller.

Medical instrument system 200 further includes a tracking system 230 for determining the position, orientation, speed, velocity, pose, and/or shape of distal end 218 and/or of one or more segments 224 along flexible body 216 using one or more sensors and/or imaging devices as described in further detail below. The entire length of flexible body 216, between distal end 218 and proximal end 217, may be effectively divided into segments 224. Tracking system 230 may optionally be implemented as hardware, firmware, software or a combination thereof which interact with or are otherwise executed by one or more computer processors, which may include the processors of control system 112 in FIG. 1.

Tracking system 230 may optionally track distal end 218 and/or one or more of the segments 224 using a shape sensor 222. Shape sensor 222 may optionally include an optical fiber aligned with flexible body 216 (e.g., provided within an interior channel (not shown) or mounted externally). In one embodiment, the optical fiber has a diameter of approximately 200 μm. In other embodiments, the dimensions may be larger or smaller. The optical fiber of shape sensor 222 forms a fiber optic bend sensor for determining the shape of flexible body 216. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some embodiments may employ other suitable strain sensing techniques, such as Rayleigh scattering, Raman scattering, Brillouin scattering, and Fluorescence scattering. In some embodiments, the shape of the elongate device may be determined using other techniques. For example, a history of the distal end pose of flexible body 216 can be used to reconstruct the shape of flexible body 216 over the interval of time. In some embodiments, tracking system 230 may optionally and/or additionally track distal end 218 using a position sensor system 220. Position sensor system 220 may be a component of an EM sensor system with position sensor system 220 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some embodiments, position sensor system 220 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

In some embodiments, tracking system 230 may alternately and/or additionally rely on historical pose, position, or orientation data stored for a known point of an instrument system along a cycle of alternating motion, such as breathing. This stored data may be used to develop shape information about flexible body 216. In some examples, a series of positional sensors (not shown), such as electromagnetic (EM) sensors similar to the sensors in position sensor 220 may be positioned along flexible body 216 and then used for shape sensing. In some examples, a history of data from one or more of these sensors taken during a procedure may be used to represent the shape of elongate device 202, particularly if an anatomic passageway is generally static.

Flexible body 216 includes a channel 221 sized and shaped to receive a medical instrument 226. FIG. 2B is a simplified diagram of flexible body 216 with medical instrument 226 extended according to some embodiments. In some embodiments, medical instrument 226 may be used for procedures such as surgery, biopsy, ablation, illumination, irrigation, or suction. Medical instrument 226 can be deployed through channel 221 of flexible body 216 and used at a target location within the anatomy. Medical instrument 226 may include, for example, image capture probes, biopsy instruments, laser ablation fibers, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. In various embodiments, medical instrument 226 is a biopsy instrument, which may be used to remove sample tissue or a sampling of cells from a target anatomic location. Medical instrument 226 may be used with an image capture probe also within flexible body 216. In various embodiments, medical instrument 226 may be an image capture probe that includes a distal portion with a stereoscopic or monoscopic camera at or near distal end 218 of flexible body 216 for capturing images (including video images) that are processed by a visualization system 231 for display and/or provided to tracking system 230 to support tracking of distal end 218 and/or one or more of the segments 224. The image capture probe may include a cable coupled to the camera for transmitting the captured image data. In some examples, the image capture instrument may be a fiber-optic bundle, such as a fiberscope, that couples to visualization system 231. The image capture instrument may be single or multi-spectral, for example capturing image data in one or more of the visible, infrared, and/or ultraviolet spectrums. Alternatively, medical instrument 226 may itself be the image capture probe. Medical instrument 226 may be advanced from the opening of channel 221 to perform the procedure and then retracted back into the channel when the procedure is complete. Medical instrument 226 may be removed from proximal end 217 of flexible body 216 or from another optional instrument port (not shown) along flexible body 216.

Medical instrument 226 may additionally house cables, linkages, or other actuation controls (not shown) that extend between its proximal and distal ends to controllably the bend distal end of medical instrument 226. Steerable instruments are described in detail in U.S. Pat. No. 7,316,681 (filed on Oct. 4, 2005) (disclosing “Articulated Surgical Instrument for Performing Minimally Invasive Surgery with Enhanced Dexterity and Sensitivity”) and U.S. patent application Ser. No. 12/286,644 (filed Sep. 30, 2008) (disclosing “Passive Preload and Capstan Drive for Surgical Instruments”), which are incorporated by reference herein in their entireties.

Flexible body 216 may also house cables, linkages, or other steering controls (not shown) that extend between drive unit 204 and distal end 218 to controllably bend distal end 218 as shown, for example, by broken dashed line depictions 219 of distal end 218. In some examples, at least four cables are used to provide independent “up-down” steering to control a pitch of distal end 218 and “left-right” steering to control a yaw of distal end 281. Steerable elongate devices are described in detail in U.S. patent application Ser. No. 13/274,208 (filed Oct. 14, 2011) (disclosing “Catheter with Removable Vision Probe”), which is incorporated by reference herein in its entirety. In embodiments in which medical instrument system 200 is actuated by a teleoperational assembly, drive unit 204 may include drive inputs that removably couple to and receive power from drive elements, such as actuators, of the teleoperational assembly. In some embodiments, medical instrument system 200 may include gripping features, manual actuators, or other components for manually controlling the motion of medical instrument system 200. Elongate device 202 may be steerable or, alternatively, the system may be non-steerable with no integrated mechanism for operator control of the bending of distal end 218. In some examples, one or more lumens, through which medical instruments can be deployed and used at a target surgical location, are defined in the walls of flexible body 216.

In some embodiments, medical instrument system 200 may include a flexible bronchial instrument, such as a bronchoscope or bronchial catheter, for use in examination, diagnosis, biopsy, or treatment of a lung. Medical instrument system 200 is also suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from tracking system 230 may be sent to a navigation system 232 where it is combined with information from visualization system 231 and/or the preoperatively obtained models to provide the physician or other operator with real-time position information. In some examples, the real-time position information may be displayed on display system 110 of FIG. 1 for use in the control of medical instrument system 200. In some examples, control system 116 of FIG. 1 may utilize the position information as feedback for positioning medical instrument system 200. Various systems for using fiber optic sensors to register and display a surgical instrument with surgical images are provided in U.S. patent application Ser. No. 13/107,562, filed May 13, 2011, disclosing, “Medical System Providing Dynamic Registration of a Model of an Anatomic Structure for Image-Guided Surgery,” and PCT Publication WO 2016/1033596 (filed May 20, 2016) (disclosing “Systems and Methods of Registration for Image Guided Surgery”), which are incorporated by reference herein in their entirety.

In some examples, medical instrument system 200 may be teleoperated within medical system 100 of FIG. 1. In some embodiments, manipulator assembly 102 of FIG. 1 may be replaced by direct operator control. In some examples, the direct operator control may include various handles and operator interfaces for hand-held operation of the instrument.

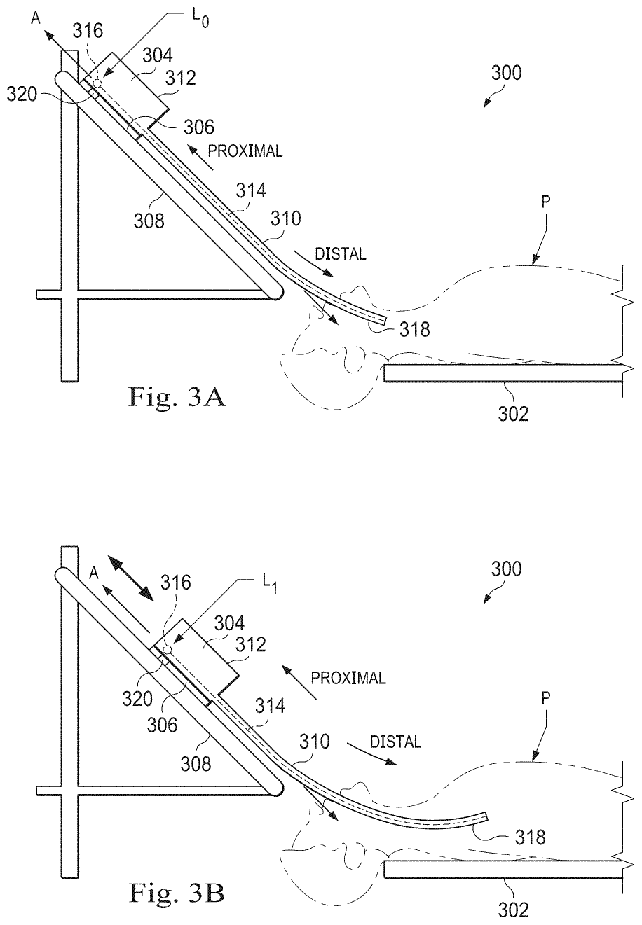

FIGS. 3A and 3B are simplified diagrams of side views of a patient coordinate space including a medical instrument mounted on an insertion assembly according to some embodiments. As shown in FIGS. 3A and 3B, a surgical environment 300 includes a patient P is positioned on the table T of FIG. 1. Patient P may be stationary within the surgical environment in the sense that gross patient movement is limited by sedation, restraint, and/or other means. Cyclic anatomic motion including respiration and cardiac motion of patient P may continue, unless patient is asked to hold his or her breath to temporarily suspend respiratory motion. Accordingly, in some embodiments, data may be gathered at a specific, phase in respiration, and tagged and identified with that phase. In some embodiments, the phase during which data is collected may be inferred from physiological information collected from patient P. Within surgical environment 300, a point gathering instrument 304 is coupled to an instrument carriage 306. In some embodiments, point gathering instrument 304 may use EM sensors, shape-sensors, and/or other sensor modalities. Instrument carriage 306 is mounted to an insertion stage 308 fixed within surgical environment 300. Alternatively, insertion stage 308 may be movable but have a known location (e.g., via a tracking sensor or other tracking device) within surgical environment 300. Instrument carriage 306 may be a component of a manipulator assembly (e.g., manipulator assembly 102) that couples to point gathering instrument 304 to control insertion motion (i.e., motion along the A axis) and, optionally, motion of a distal end 318 of an elongate device 310 in multiple directions including yaw, pitch, and roll. Instrument carriage 306 or insertion stage 308 may include actuators, such as servomotors, (not shown) that control motion of instrument carriage 306 along insertion stage 308.

Elongate device 310 is coupled to an instrument body 312. Instrument body 312 is coupled and fixed relative to instrument carriage 306. In some embodiments, an optical fiber shape sensor 314 is fixed at a proximal point 316 on instrument body 312. In some embodiments, proximal point 316 of optical fiber shape sensor 314 may be movable along with instrument body 312 but the location of proximal point 316 may be known (e.g., via a tracking sensor or other tracking device). Shape sensor 314 measures a shape from proximal point 316 to another point such as distal end 318 of elongate device 310. Point gathering instrument 304 may be substantially similar to medical instrument system 200.

A position measuring device 320 provides information about the position of instrument body 312 as it moves on insertion stage 308 along an insertion axis A. Position measuring device 320 may include resolvers, encoders, potentiometers, and/or other sensors that determine the rotation and/or orientation of the actuators controlling the motion of instrument carriage 306 and consequently the motion of instrument body 312. In some embodiments, insertion stage 308 is linear. In some embodiments, insertion stage 308 may be curved or have a combination of curved and linear sections.

FIG. 3A shows instrument body 312 and instrument carriage 306 in a retracted position along insertion stage 308. In this retracted position, proximal point 316 is at a position L₀ on axis A. In this position along insertion stage 308 an A component of the location of proximal point 316 may be set to a zero and/or another reference value to provide a base reference to describe the position of instrument carriage 306, and thus proximal point 316, on insertion stage 308. With this retracted position of instrument body 312 and instrument carriage 306, distal end 318 of elongate device 310 may be positioned just inside an entry orifice of patient P. Also in this position, position measuring device 320 may be set to a zero and/or the another reference value (e.g., I=0). In FIG. 3B, instrument body 312 and instrument carriage 306 have advanced along the linear track of insertion stage 308 and distal end 318 of elongate device 310 has advanced into patient P. In this advanced position, the proximal point 316 is at a position L₁ on the axis A. In some examples, encoder and/or other position data from one or more actuators controlling movement of instrument carriage 306 along insertion stage 308 and/or one or more position sensors associated with instrument carriage 306 and/or insertion stage 308 is used to determine the position L₁ of proximal point 316 relative to position L₀. In some examples, position L₁ may further be used as an indicator of the distance or insertion depth to which distal end 318 of elongate device 310 is inserted into the passageways of the anatomy of patient P.

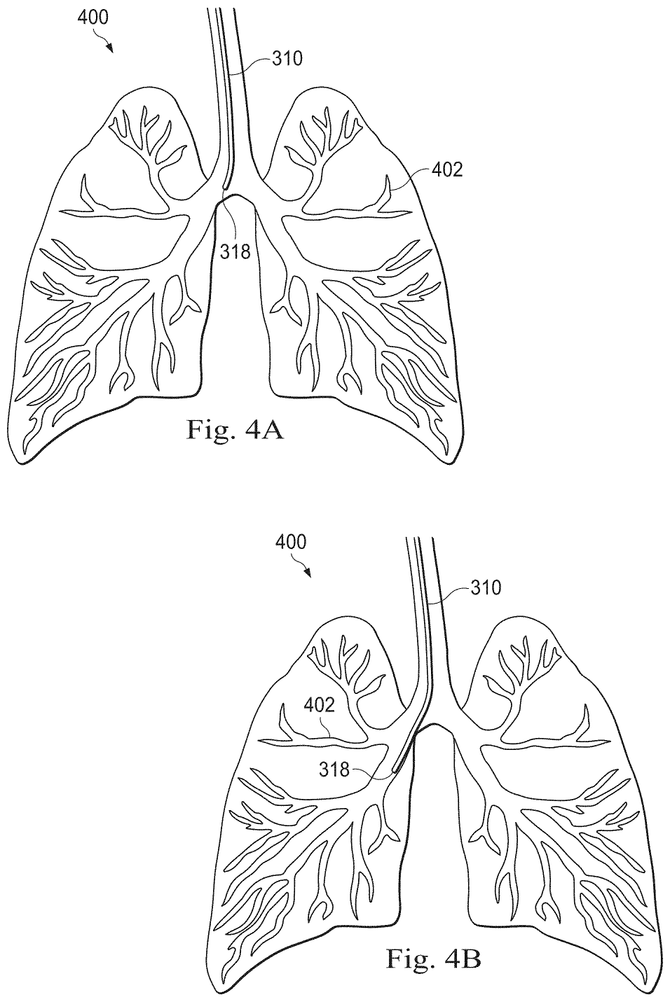

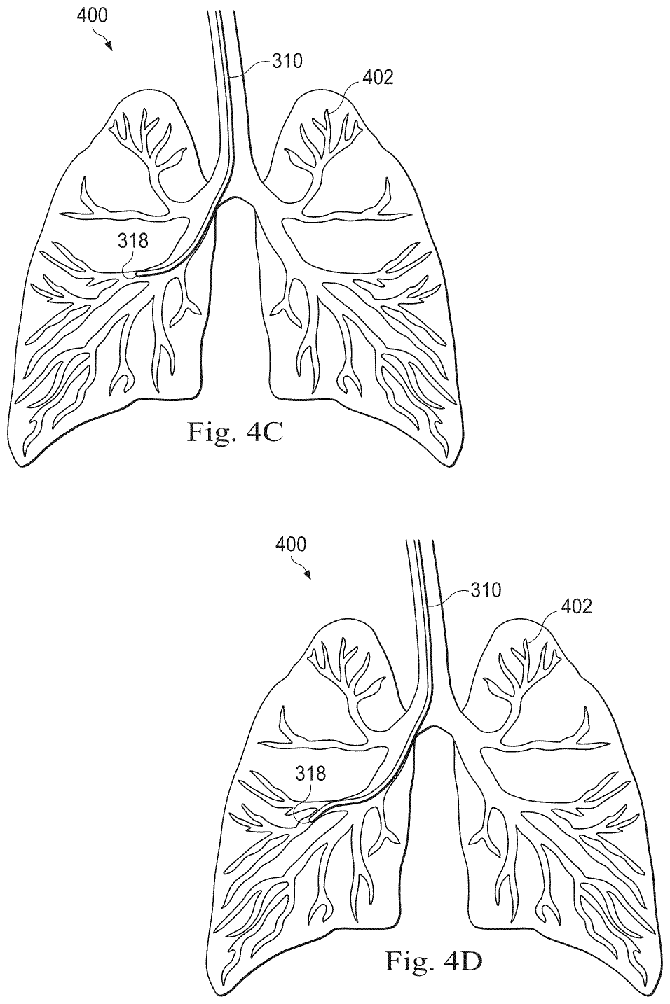

FIGS. 4A, 4B, 4C, and 4D illustrate the advancement of elongate device 310 of FIGS. 3A and 3B through anatomic passageways 402 of the lungs 400 of the patient P of FIGS. 1 and 3A and 3B. These passageways 402 include the trachea and the bronchial tubes. As the elongate device 310 is advanced with the carriage 306 moving along the insertion stage 308, the operator O may steer the distal end 318 of elongate device 310 to navigate through the anatomic passageways 402. In navigating through the anatomic passageways 402, elongate device 310 assumes a shape that may be “read” by the shape sensor 314 extending within the elongate device 310.

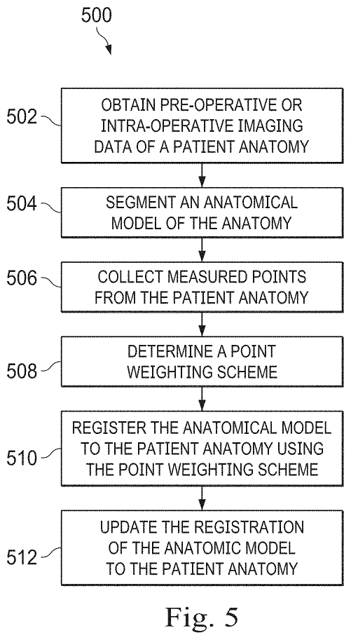

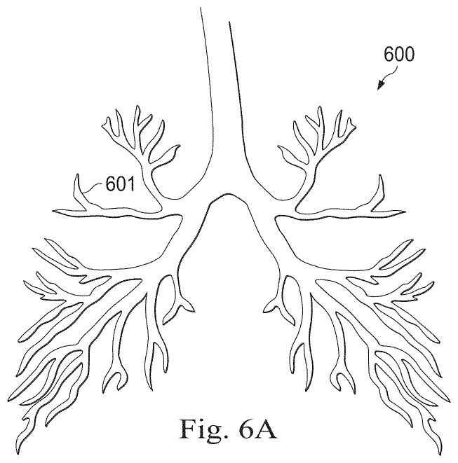

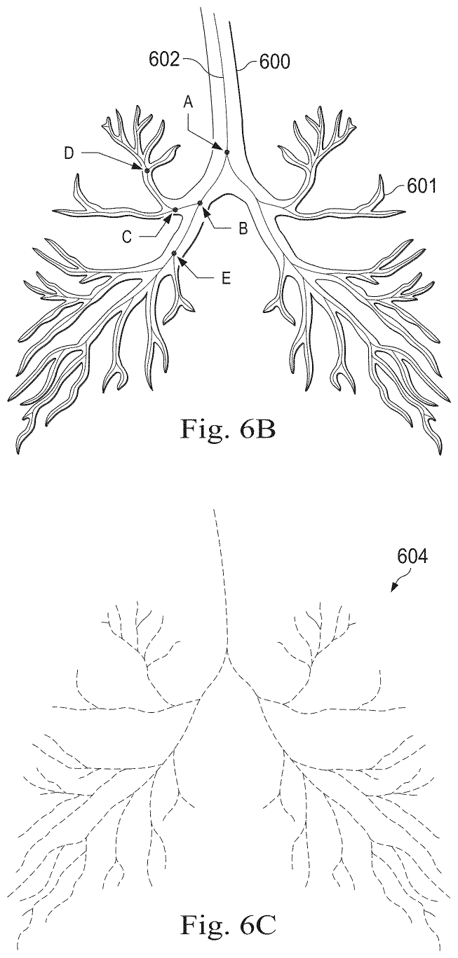

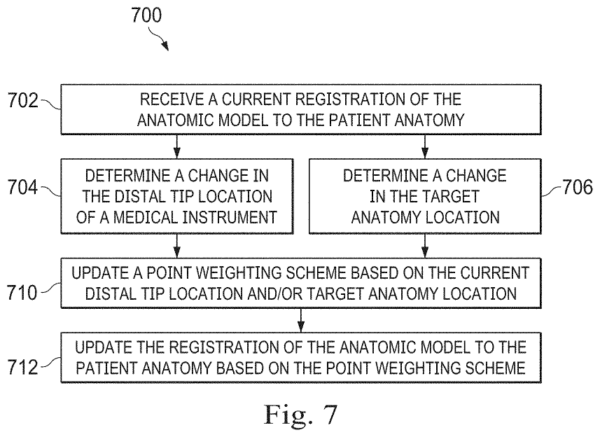

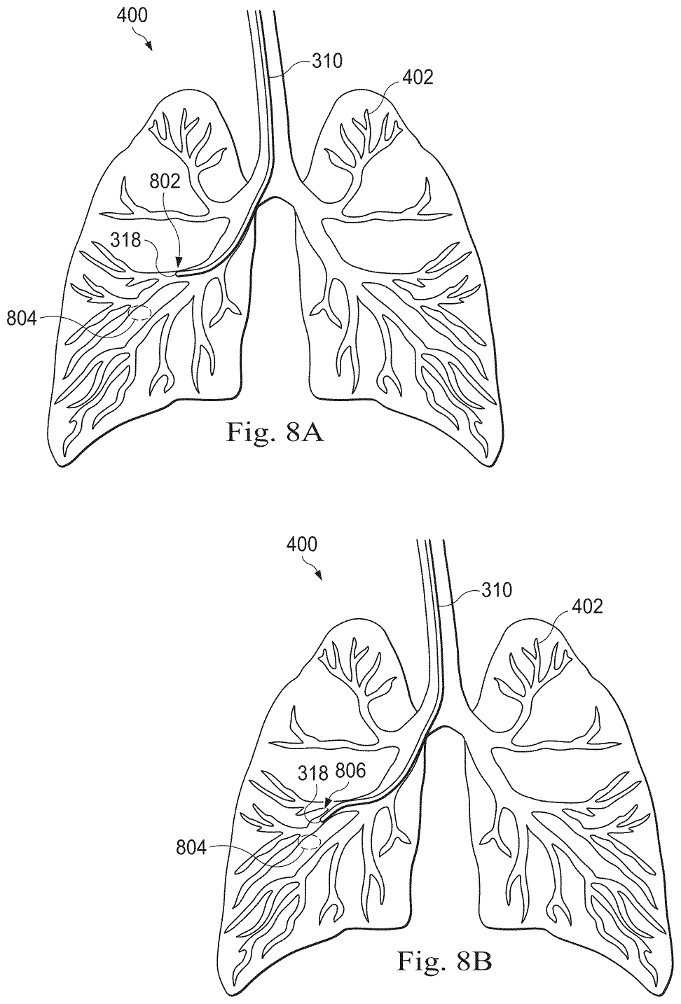

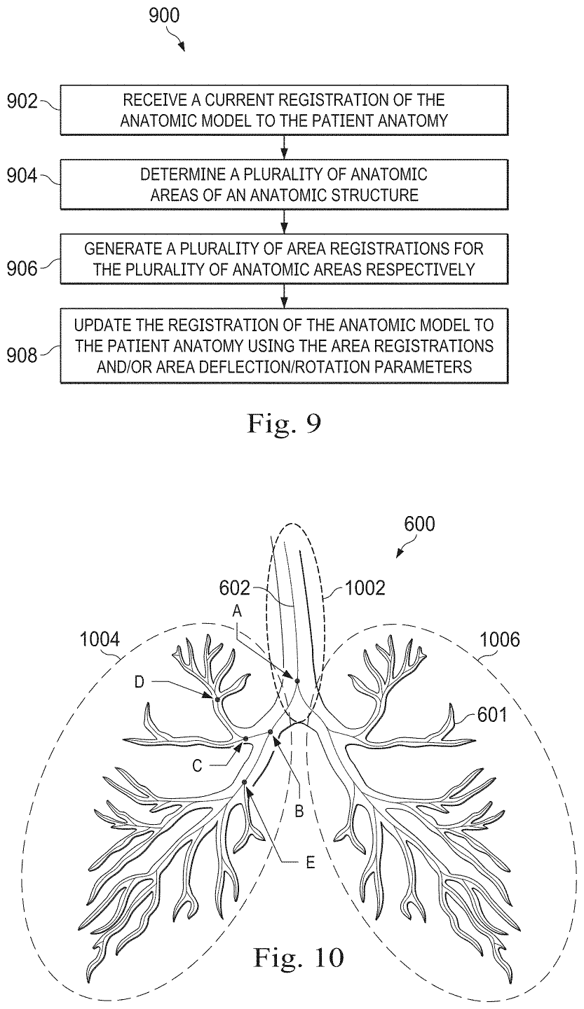

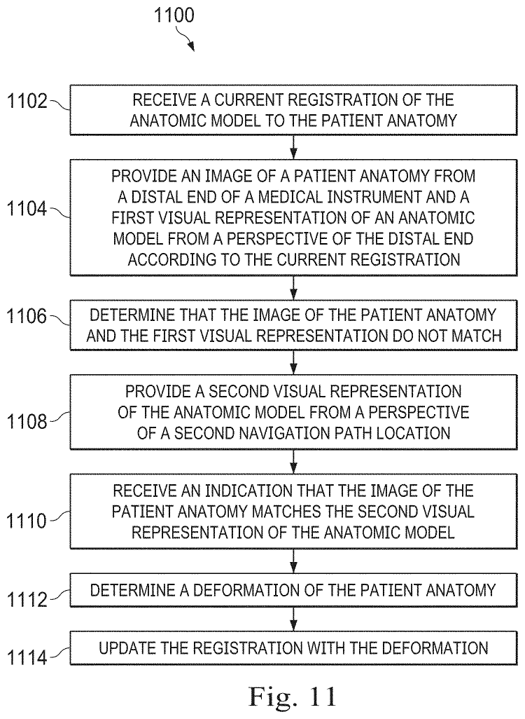

Referring to FIGS. 5, 6A, 6B, 6C, 7, 8A, 8B, 9, 10, 11, and 12, various embodiments for image-guided surgical procedures using weighted and/or non-rigid registration are described. FIG. 5 is a flowchart illustrating a general method 500 for use in an image-guided surgical procedure. FIGS. 6A, 6B, and 6C illustrate segmentation processes of the general method 500 that generates a model of human lungs for registration. FIGS. 7, 8A, and 8B illustrate a method for performing a weighted registration based on a real-time location of a distal end of an elongated device during insertion within a patient anatomy. FIGS. 9 and 10 illustrate a method for performing a non-rigid registration taking into account deformation, deflection, and orientation of different anatomic areas of an anatomic structure. FIGS. 11, 12, and 13 illustrate a method for performing a registration by matching a patent anatomy image with a visual representation of the anatomic model.

FIG. 5 is a flowchart illustrating a general method 500 for use in an image-guided surgical procedure. The method 500 is illustrated in FIG. 5 as a set of operations or processes 502 through 512. Not all of the illustrated processes 502 through 512 may be performed in all embodiments of method 500. Additionally, one or more processes that are not expressly illustrated in FIG. 5 may be included before, after, in between, or as part of the processes 502 through 512. In some embodiments, one or more of the processes may be implemented, at least in part, in the form of executable code stored on non-transitory, tangible, machine-readable media that when run by one or more processors (e.g., the processors of control system 112) may cause the one or more processors to perform one or more of the processes.

At a process 502, pre-operative or intra-operative image data is obtained from imaging technology such as, computed tomography (CT), magnetic resonance imaging (MRI), fluoroscopy, thermography, ultrasound, optical coherence tomography (OCT), thermal imaging, impedance imaging, laser imaging, or nanotube X-ray imaging. The pre-operative or intra-operative image data may correspond to two-dimensional, three-dimensional, or four-dimensional (including e.g., time based or velocity based information) images. For example, the image data may represent the human lungs 400 of FIGS. 4A-4D.

At a process 504, a computer system either operating alone or in combination with manual input is used to convert the recorded images into a segmented two-dimensional or three-dimensional composite representation or model of a partial or an entire anatomic organ or anatomic region. For example, FIG. 6A illustrates a segmented model 600 of the lungs 400 of FIGS. 4A-4D. Due to naturally occurring limitations or to limitations set by an operator, the segmented model 600 may not include all of the passageways present within the human lungs, but includes some passageways 601. For example, relatively narrow and/or distal passageways of the lungs may not be fully included in the segmented model 600. The segmented model 600 may be a three-dimensional model, such as a mesh model or another suitable model, that includes the walls defining the interior lumens or passageways of the lungs. In general, the model provides a mechanism or means for distinguishing between points within a region of anatomy and points outside the region of anatomy. The composite representation and the image data set describe the various locations and shapes of the passageways and their connectivity and may omit undesired portions of the anatomy included in the pre-operative or intra-operative image data. In some embodiments, the model 600 may include specifically desired features, such as a suspected tumor or other tissue portion of interest.

During the segmentation process the images are partitioned into segments or elements (e.g., pixels or voxels) that share certain characteristics or computed properties such as color, density, intensity, and texture. This segmentation process results in a two- or three-dimensional reconstruction that forms a model of the target anatomy based on the obtained image, like the model 600. To represent the model, the segmentation process may delineate sets of voxels representing the target anatomy and then apply a function, such as marching cube function, to generate a 3D surface that encloses the voxels. The model may be made by generating a mesh, volume, or voxel map. This model may be shown in the display 110 to aid the operator O in visualizing the anatomy, such as the interior passageways of the lungs.

Additionally or alternatively, the model may include a centerline model that includes a set of interconnected line segments or points extending through the centers of the modeled passageways. FIG. 6B shows an exemplary centerline model 602 derived from the model 600 or directly from the imaging data. The centerline segmented model 602 may include a set of three-dimensional straight lines or a set of curved lines that correspond to the approximate center of the passageways contained in the segmented model 602. The higher the resolution of the model, the more accurately the set of straight or curved lines will correspond to the center of the passageways. Representing the lungs with the centerline segmented model 602 may provide a smaller set of data that is more efficiently processed by one or more processors or processing cores than the data set of the segmented model 602, which represents the walls of the passageways of model 600. In this way the functioning of the control system 112 may be improved.

As shown in FIG. 6B, the centerline segmented model 602 includes several branch points, some of which are highlighted for visibility in FIG. 6B. The branch points A, B, C, D, and E are shown at each of several of the branch points. The branch point A may represent the point in the model at which the trachea divides into the left and right principal bronchi. The right principal bronchus may be identified in the centerline segment model 602 as being located between branch points A and B. Similarly, secondary bronchi are identified by the branch points B and C and between the branch points B and E. Another generation may be defined between branch points C and D. Each of these generations may be associated with a representation of the diameter of the lumen of the corresponding passageway. In some embodiments, the model 602 may include an average diameter value of each segmented generation. The average diameter value may be a patient-specific value or a more general value derived from multiple patients.

Where the model includes a centerline model including a set of interconnected line segments, those line segments may be converted to a cloud or set of points 604, referred to as model points, which are represented by the dashed lines of FIG. 6C. By converting the line segments into points, a desired quantity of model points corresponding to the interconnected line segments can be selected manually or automatically to represent the centerline model 602 (and thereby the model 600) during a registration process. In data, each of the points of the set of model points 604 may include coordinates such as a set of X_{M}, Y_{M}, and Z_{M}, coordinates, or other coordinates that identify the location of each point in the three-dimensional model space. In some embodiments, each of the points may include a generation identifier that identifies which passageway generation the points are associated with and/or a diameter or radius value associated with that portion of the centerline segmented model 602. In some embodiments, information describing the radius or diameter associated with a given point may be provided as part of a separate data set.

After the centerline segmented model 602 is generated and stored in data as the set of points 604 shown in FIG. 6C, the model points 604 may be retrieved from data storage for use in an image-guided surgical procedure. In order to use the centerline segmented model 602 and the model 600 in the image-guided surgical procedure, the model points 604 may be registered to associate the modeled passageways in the model 600 with the patient's actual anatomy as present in a surgical environment.

Returning to FIG. 5, at a process 506, measured points may be obtained from patient anatomy that corresponds to the anatomic model, as shown in FIGS. 3A-3B and 4A-4D. The measured points are associated with a patient space, and may also be referred to as patient space points. Measured points may be generated by driving through anatomy and/or touching landmarks in the anatomy, and tracking position based on electromagnetic coils and/or a sensor system (e.g., the sensor system 108).

At a process 508, a point weighting scheme is determined for registering the anatomic model to the patient anatomy. Weightings may be assigned to measured points, model points, and/or matches between pairs of a measured point and a model point. In embodiments where weightings are assigned to measured points, the weighting may be determined independently from the model. For example, weightings may be based solely on the depth of insertion of an elongated device as measured by an insertion or position sensor as described in reference to FIGS. 3A-3B. In this example, measured points may be weighted low if the elongated device has been inserted a small distance and weighted higher with deeper insertion. In some embodiments, the measured point may be weighted low if an elongate device is inserted a relatively large distance. In embodiments where weightings are assigned to model points, the weightings may be determined solely based on the model. For example, points that do not connect to other points may be considered noise and be weighted a very low or zero value. In embodiments where weightings are assigned to matches between the measured points and the corresponding model points, the model point and corresponding measured point are considered a match point or a match and is given a weight. In some embodiments, the point weighting scheme for the match is determined based on proximity of the match to a target anatomic location. For example, a weight of a match is determined based on a distance between the match and the target anatomic location. In that example, a match associated with a model point closer to the target anatomic location may have a greater weight. In another example, a weight of a match is determined based on a distance between that match's associated model point and a predetermined navigation path to the target anatomic location. In that example, a match with a model point closer to the predetermined navigation path to the target anatomic location may have a greater weight. In some embodiments, the weights of the matches are determined using a sliding weight scale. In some embodiments, a weight having a value of zero may be assigned to a match when the distance between the model point of that match and the target anatomic location/predetermined navigation path to target anatomic location is greater than a predetermined target anatomic location distance threshold. In those examples, the matches having a weight of zero may be discarded during a subsequent registration process.

At a process 510, the anatomic model data of a model space is registered to the patient anatomy of a patient space (or vice versa) prior to and/or during the course of an image-guided surgical procedure on the patient. In some embodiments, the point weighting scheme is used to apply weights to the measured points, the model points, and/or the matches between the measured points and the corresponding model points during the registration. Generally, registration involves the matching of measured points to model points of the model through the use of rigid and/or non-rigid transforms. A point set registration method (e.g., iterative closest point (ICP) technique) may be used in registration processes within the scope of this disclosure. Such a point set registration method may generate a transformation that aligns the measured points (also referred to as a measured point set) and the model points (also referred to as a model point set). In some embodiments, the registration may also generate a deformation model associated with deformation of the patient anatomy associated with the measured points and/or model points.

After the process 510 in which the anatomic model is registered to the patent anatomy so that the medical instrument positioned with respect to the patient anatomy may be represented with respect to the anatomic model, the medical instrument may be advanced in a patient anatomy. As the medical instrument moves to a new location, the registration may be updated at a process 512. The updating of the registration may be performed continuously throughout a surgical procedure. In this way, changes due to patient movements (both gross movements and periodic physiological movements), patient breathing, movement of the medical instrument, and/or any other factors that may cause changes to the patient anatomy may be compensated for.

Other registration methods for use with image-guided surgery often involve the use of technologies based on electromagnetic or impedance sensing. Metallic objects or certain electronic devices used in the surgical environment may create disturbances that impair the quality of the sensed data. Other methods of registration may obstruct the clinical workflow. Some embodiments of the systems and methods described herein perform registration based upon ICP, or another point set registration algorithm, and the calibrated movement of a point gathering instrument with a fiber optic shape sensor, thus eliminating or minimizing disruptions in the surgical environment. Other registration techniques may be used to register a set of measured points to a pre-operative model or a model obtained using another modality. In the embodiments described below, EM sensors on the patient and the instrument and optical tracking systems for the instrument may be eliminated.

Referring to FIGS. 7, 8A, and 8B, the process for updating the registration (e.g., process 512 of FIG. 5) may include a method 700 to provide an improved registration by using a weighting scheme based on the distal end location of the elongate device and/or the target anatomic location.

Referring to FIG. 7, the method 700 in FIG. 7 is illustrated as a set of operations or processes 702 through 712. Not all of the illustrated processes 702 through 712 may be performed in all embodiments of method 700. Additionally, one or more processes that are not expressly illustrated in FIG. 7 may be included before, after, in between, or as part of the processes 702 through 712. In some embodiments, one or more of the processes may be implemented, at least in part, in the form of executable code stored on non-transitory, tangible, machine-readable media that when run by one or more processors (e.g., the processors of control system 112) may cause the one or more processors to perform one or more of the processes.

The method 700 begins at a process 702, where a current registration of the anatomic model to the patent anatomy is received. In an example, the current registration is the registration generated at a registration process 510 of FIG. 5 prior to driving the elongate device toward the target anatomic location. In another example, referring to FIG. 8A, the elongate device 310 is driven toward a target anatomic location 804. In that example, in the process 702, the distal end 318 of the elongate device 310 is at a distal end location 802. The current registration may be generated in an update registration process 512 of FIG. 5 using a point weighting scheme based on the distal end location 802 and/or the target anatomic location 804.

At process 704, it is determined that there is a change in the location of the distal end 318 of the elongate device 310. Referring to the example of FIG. 8B, at process 704, it is determined that the distal end 318 of the elongate device 310 is advanced from the distal end location 802 to the distal end location 806.

While in the example of FIG. 8B, the target anatomic location 804 remains the same, in some embodiments, the target anatomic location may shift (e.g., based on an input of an operator). In those embodiments, at process 706, it is determined that the target anatomic location is moved to an updated target anatomic location.

At process 710, a point weighting scheme may be updated based on the changed distal end location and/or the changed (current) target anatomic location. Specifically, weights updated based on the changed distal end location and/or the changed target anatomic location may be assigned to measured points (e.g., measured points collected at process 506 of FIG. 5 and/or new measured points collected while the elongate device is driven toward the target anatomic location) of the patient anatomy. In some embodiments, a weight of a measured point is determined based on a distance between that measured point and the current distal end location. In that example, a measured point closer to the current distal end location may have a greater weight. In some embodiments, the weights of the measured points are determined using a sliding weight scale based on the distances between the measured points and the current distal end location. In some embodiments, a weight having a value of zero may be assigned to a measured point when the distance between that measured point and the current distal end location is greater than a predetermined distal end distance threshold. In those examples, the measured points having a weight of zero may be discarded during a subsequent registration process.

In some embodiments, a weight of a match may be alternatively or additionally determined based on a distance between its associated model point and the target anatomic location and/or a distance between the model point and a predetermined navigation path to the target anatomic location, for example, as discussed above with reference to process 508 of FIG. 5.

At process 712, the registration of the anatomic model to the patent anatomy is performed again using the point weighting scheme generated in the process 710. As such, as the elongate device 310 is driven toward the target anatomic location, a registration may be updated continuously based on the current distal end location and target anatomic location.

Referring to FIGS. 9 and 10, in some embodiments, the process for updating the registration (e.g., process 512 of FIG. 5) may include a method 900 to provide an improved registration by taking into account the deformation, deflection, and rotation of different areas of an anatomic structure. In various embodiments, the anatomic structure may be divided into a plurality of anatomic areas (e.g., based on the rigidity of the anatomic areas). In some examples, a local registration may be performed for each of those anatomic areas to generate a corresponding area registration. Those area registrations may then be used to update the registration of the anatomic model to the measured points. In some examples, the registration method may use deflection and rotation of different anatomic areas (e.g., with a global registration of the anatomic structure or local registrations of the anatomic areas), and generate deflection and/or rotation parameter estimates for each anatomic area.

Referring to FIG. 9, the method 900 in FIG. 9 is illustrated as a set of operations or processes 902 through 908. Not all of the illustrated processes 902 through 908 may be performed in all embodiments of method 900. Additionally, one or more processes that are not expressly illustrated in FIG. 9 may be included before, after, in between, or as part of the processes 902 through 908. In some embodiments, one or more of the processes may be implemented, at least in part, in the form of executable code stored on non-transitory, tangible, machine-readable media that when run by one or more processors (e.g., the processors of control system 112) may cause the one or more processors to perform one or more of the processes.

The method 900 begins at a process 902, where a current registration of the anatomic model to the patent anatomy is received. In an example, the current registration is the registration generated at a registration process 510 of FIG. 5 prior to driving the elongate device toward the target anatomic location. In another example, the current registration is a registration generated at a registration process 512 of FIG. 5 during driving the elongate device toward the target anatomic location.

In process 904, an anatomic structure is divided into a plurality of anatomic areas. In various embodiments, the lungs may be divided into any suitable number of anatomic areas. Referring to the example of FIG. 10, illustrated is an anatomic model 600 of human lungs of a patient. In the example of FIG. 10, it is determined that the left lung and right lung of the human lungs tend to deform at the main carina (e.g., around branch point A at which the trachea divides into the left and right principal bronchi), while the individual structures of the left lung and the right lung are preserved. Accordingly, the anatomic model 600 is divided into anatomic areas 1002, 1004, and 1006 based on the main carina. The anatomic area 1002 includes the central area of the lungs, the anatomic area 1004 includes the right lung, and the anatomic area 1006 includes the left lung. In another example, the lungs of a patient may be divided into six anatomic areas including a central area 1002, a superior lobe area of the right lung, a middle lobe area of the right lung, an inferior lobe area of the right lung, a superior lobe area of the left lung, and an inferior lobe area of the left lung.

At process 906, each anatomic area of the anatomic structure is registered with the corresponding model area of the anatomic model to generate a local registration. For example, measured points (e.g., collected during a process 506 and/or while the elongate device is driving toward the target anatomic location) may be divided into sets of measured points corresponding to the anatomic areas based on the current registration and the anatomic model. In the example of FIG. 10, for each of the anatomic areas 1002, 1004, and 1006, the subset of model points in the corresponding anatomic area is registered to the subsets of measured points in that anatomic area to generate an area registration. The registration method may include a point set registration algorithm such as an iterative closest point (ICP) technique, or another registration algorithm.

At process 908, the registration of the anatomic model to the patient anatomy is updated using those area registrations. In some embodiments, the updated registration includes three separate area registrations. In those embodiments, a point (e.g., a distal end location of the elongate device) in the patient space may be transformed to the model space strictly based on the anatomic area of that point. For example, a point in the anatomic area 1002 of the patient space is transformed to the model space using the area registration for the anatomic area 1002, and a point in the anatomic area 1004 of the patient space is transformed to the model space using the area registration for the anatomic area 1004. In such embodiments, when a distal end of the elongate device is driven through a transition area of two adjacent anatomic areas (e.g., a transition area of adjacent anatomic areas 1002, and 1004, a transition area of adjacent anatomic areas 1002 and 1006), there may be jumps in one or more images displayed to an operator (e.g., using a display system 110). Those images may be used to facilitate guidance of the navigation and/surgery for the operator. In an example, the images include a virtual navigational image including a virtual image of the elongate device within the patent anatomy from an external viewpoint. In another example, the images include an internal view of a portion of the anatomic model from a perspective of a distal end of the elongate device registered to the anatomic model. As such, the jumps in those images may cause disturbance and uncertainty to the image guided surgery. As such, in some embodiments, at process 908, the registration blends the separate area registrations in a transition area of adjacent anatomic areas, such that the transition between adjacent anatomic areas is smoothed.

In some embodiments, at process 908, the registration takes into account the deflection and/or rotation of each anatomic area. In an example, for each anatomic area, a deflection parameter and a rotation parameter are estimated. Various optimization methods (e.g., stochastic parameter variation and minimization or any other suitable minimization method) may be used in the registration process. In some examples, the optimization method may include cost functions for minimizing point match residues and penalizing excessive or unnaturally large deflections and/or rotations. In some examples, the optimization method may use the quantity and quality of the measured points (e.g., the total measured points, the subset of measured points for each anatomic area) to avoid over-fitting the anatomic model. By taking into account various rigidity, deflection, and rotation of separate anatomic areas of an anatomic structure, the registration of the anatomic model to the measured points is improved.

Referring to FIGS. 11, 12, and 13, in some embodiments, the process for updating the registration (e.g., process 512 of FIG. 5) may include a method 1100 to provide an improved registration by matching an image of the patient anatomy with a rendered internal view of an anatomic model.

Referring to FIG. 11, the method 1100 in FIG. 11 is illustrated as a set of operations or processes 1102 through 1114. Not all of the illustrated processes 1102 through 1114 may be performed in all embodiments of method 1100. Additionally, one or more processes that are not expressly illustrated in FIG. 11 may be included before, after, in between, or as part of the processes 1102 through 1114. In some embodiments, one or more of the processes may be implemented, at least in part, in the form of executable code stored on non-transitory, tangible, machine-readable media that when run by one or more processors (e.g., the processors of control system 112) may cause the one or more processors to perform one or more of the processes.

The method 1100 begins at a process 1102, where a current registration of the anatomic model to the patent anatomy is received. In an example, the current registration is the registration generated at a registration process 510 of FIG. 5 prior to driving the elongate device toward the target anatomic location. In another example, the current registration is a registration generated at a registration process 512 of FIG. 5 during driving the elongate device toward the target anatomic location.

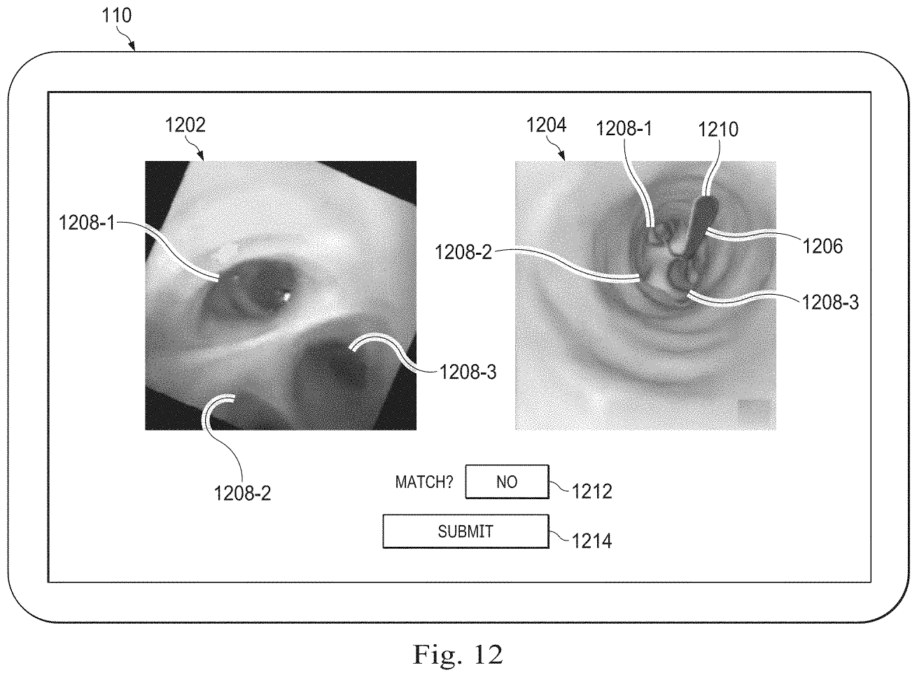

At process 1104, a concurrent or real-time image (e.g., captured by a visualization system) of the patient anatomy from the perspective of the distal end of the elongate device and a first visual representation of an internal view of the anatomic model from a perspective of the distal end are provided. Referring to the example of FIG. 12, a display system 110 displays a concurrent or real-time image 1202 of the patient anatomy from the distal end of the elongate device and a first visual representation 1204 of an internal view of the anatomic model from a perspective of the distal end of the elongate device.

As shown in the example of FIG. 12, the concurrent or real-time image 1202 includes passageways 1208-1, 1208-2, and 1208-3 of a lung of the patent. The first visual representation 1204 illustrates a navigation path 1206 to a target anatomic location and the rendered model images for passageways 1208-1, 1208-2, and 1208-3 based on the anatomic model. In some embodiments, the current distal end location of the elongate device is registered to a first navigation path location 1210 based on the current registration (e.g., received at the process 1102). The first visual representation 1204 is generated by generating an internal view of the anatomic model from the perspective of the first navigation path location 1210 toward the target anatomic location along the navigation path 1206.

At process 1106, it is determined that the concurrent or real-time image 1202 and the first visual representation 1204 do not match. In the example of FIG. 12, the passageways 1208-1, 1208-2, and 1208-3 in the first visual representation 1204 is further away from the first navigation path location 1210 (corresponding to the current distal end location registered in the anatomic model using the current registration) than the passageways 1208-1, 1208-2, and 1208-3 in the real-time image 1202 from the current distal end location. In some embodiments, such a mismatch may be caused by deformation of the lungs of the patient (e.g., caused by patient movements including for example gross movements and periodic physiological movements, movement of the elongate device, etc.).

In some embodiments, such a mismatch between the concurrent or real-time image 1202 and the first visual representation 1204 is determined automatically by a control system (e.g., by performing an image processing method to compare the concurrent or real-time image 1202 and the first visual representation 1204). Alternatively, as shown in the example of FIG. 12, in some embodiments, the mismatch is determined and provided by an operator. In the example of FIG. 12, the operator determines that the concurrent or real-time image 1202 and the first visual representation 1204 do not match (e.g., using a choice 1212), and submits the mismatch determination to the control system (e.g., using a button 1214).

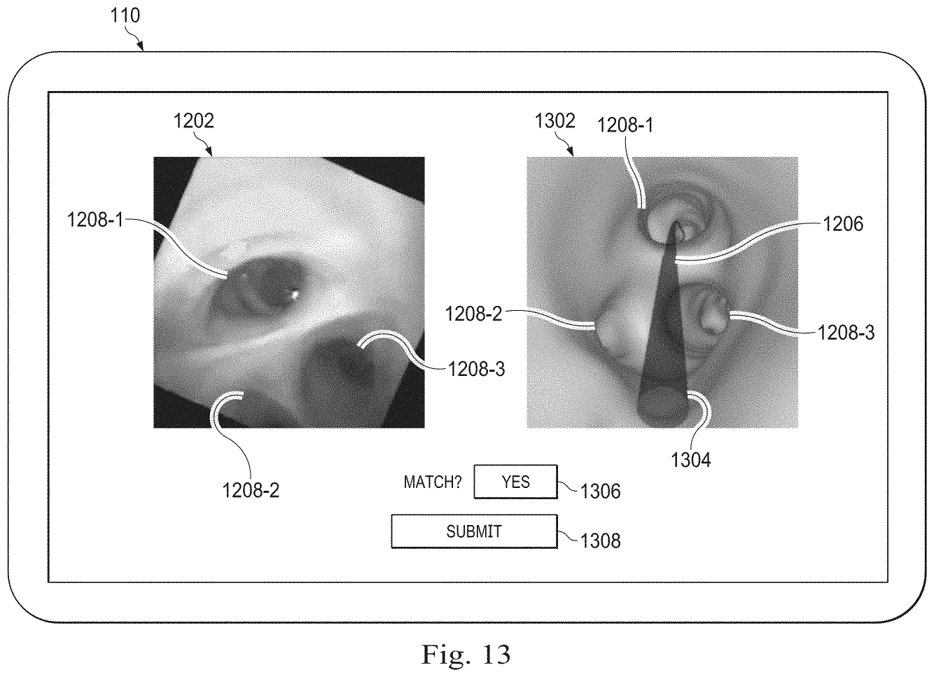

At process 1108, a second visual representation of an internal view of the anatomic model from a perspective of a second navigation path location is provided. Referring to the example of FIG. 13, the display system 110 displays the concurrent or real-time image 1202 of the patient anatomy from the distal end of the elongate device and a second visual representation 1302 of an internal view of the anatomic model from a perspective of a second navigation path location 1304. In some embodiments, the concurrent or real-time image 1202 of FIG. 13 is the same as the concurrent or real-time image 1202 of FIG. 12, as the distal end location of the elongate device remains the same during processes 1106 and 1108.

As shown in the example of FIG. 13, compared to the first navigation path location 1210, the second navigation path location 1304 is closer to the target anatomic location. As such, compared to the first visual representation 1204, the passageways 1208-1, 1208-2, and 1208-3 in the second visual representation 1302 are closer to the view point. In other examples, the second navigation path location 1304 may be further away from the target anatomic location, such that the passageways 1208-1, 1208-2, and 1208-3 in the second visual representation 1302 are further away from the view point.

In some embodiments, the second navigation path location is determined automatically by the control system. Alternatively, in some embodiments, an operator may adjust the second navigation path location along the navigation path using an input device.

At process 1110, an indication that the concurrent or real-time image and the second visual representation of the anatomic model match is received. In some embodiments, such an indication is provided by the control system after comparing the concurrent or real-time image and the second visual representation of the anatomic model. Alternatively, as shown in FIG. 13, in some embodiments, such a match indication is determined and provided by an operator. In the example of FIG. 13, the operator determines that the concurrent or real-time image 1202 and the second visual representation 1302 match (e.g., using a choice 1306), and submits the match indication to the control system (e.g., using a button 1308).

At process 1112, a deformation of the anatomic structure is determined based on the current distal end location, the current registration, and the second navigation path location. In an example, such a deformation is determined by using a model of possible lung deformations (e.g., based on breathing motion), where by using that deformation, the current distal end location and the second navigation path location have the most close fit.

At process 1114, the registration is then updated using the deformation determined at process 1112. In other words, the updated registration is improved by taking into account the determined deformation of the anatomic structure.

The systems and methods of this disclosure may be used for connected bronchial passageways of the lungs. The systems and methods may also be suited for navigation and treatment of other tissues, via natural or surgically created connected passageways, in any of a variety of anatomic systems including the colon, the intestines, the kidneys, the brain, the heart, the circulatory system, or the like. The systems and methods may also be suitable for navigation around the traceable surface of an organ. The methods and embodiments of this disclosure are also suitable for non-surgical applications.

In some exemplary embodiments, a method performed by a computing system includes accessing a set of model points of a model of an anatomic structure of a patient, the model points being associated with a model space; collecting a set of measured points of the anatomic structure of the patient, the measured points being associated with a patient space; determining a first plurality of weights for the set of measured points respectively; and registering the set of model points to the set of measured points based on the first plurality of weights to generate a registration. The set of measured points are collected during insertion of a medical instrument in the anatomic structure of the patient. The determining the first plurality of weights is based on an insertion depth of the medical instrument at the time the set of measured points are collected.

In some exemplary embodiments, a method performed by a computing system includes accessing a set of model points of a model of an anatomic structure of a patient, the model points being associated with a model space; collecting a set of measured points of the anatomic structure of the patient, the measured points being associated with a patient space; registering the set of model points with the set of measured points to generate a first registration; providing a patient anatomic image from a distal end location of a medical instrument; determining a mismatch between the patient anatomic image and a first visual representation of the model from a first navigation path location, the first navigation path location being determined based on the distal end location and the first registration; providing a second visual representation of the model from a second navigation path location different from the first navigation path location; receiving a match indication that the patient anatomic image matches the second visual representation of the model; and generating a second registration for translating the model space to the patient space based on the distal end location and the second navigation path location. In some embodiments, the match indication is provided by an operator. In some embodiments, the generating the second registration includes: determining a deformation of the anatomic structure based on the distal end location and the second navigation path location; and updating the second registration using the deformation. In some embodiments, a non-transitory machine-readable medium includes a plurality of machine-readable instructions which, when executed by one or more processors, are adapted to cause the one or more processors to perform the one or more methods described herein.

In some exemplary embodiments, a method performed by a computing system includes accessing a set of model points of a model of an anatomic structure of a patient, the model points being associated with a model space; collecting a set of measured points of the anatomic structure of the patient, the measured points being associated with a patient space; determining a set of matches between the set of model points and the set of measured points; determining a first plurality of weights for the set of matches; and registering the set of model points to the set of measured points based on the first plurality of weights to generate a first registration. In some embodiments, the first plurality of weights for the set of matches is based on a proximity of each of the matches to an anatomic target, wherein the anatomic target is associated with the model space. In some embodiments, the method further includes determining a second plurality of weights for the set of model points or the set of measured points. In some embodiments, the registering the set of model points to the set of model points is further based on the second plurality of weights. In some embodiments, the method further includes obtaining a first distal end location of a distal end of a medical instrument inserted into the anatomic structure. In some embodiments, the determining the first plurality of weights includes: for each measured point, determining a distal end distance between the measured point and the first distal end location; and assigning a weight to the measured point based on the distal end distance. In some embodiments, the assigning the weight to the measured point based on the distal end distance includes: determining that the distal end distance is greater than a predetermined distal end distance threshold; and assigning the weight having a value of zero to the measured point. In some embodiments, a first measured point has a first distance from the first distal end location, wherein a second measured point has a second distance from the first distal end location, the second distance being less than the first distance, and wherein a first weight assigned to the first measured point is less than a second weight assigned to the second measured point. In some embodiments, the method further includes detecting a movement of the distal end to a second distal end location; determining a second plurality of weights for the set of measured points respectively based on the second distal end location; and registering the set of model points to the set of measured points based on the second plurality of weights to generate a second registration. In some embodiments, the method further includes for each measured point, determining a target distance between the measured point and a target anatomic location; and assigning the weight to the measured point based on at least one of the distal end distance and the target distance.

In some exemplary embodiments, a method performed by a computing system includes accessing a set of model points of a model of an anatomic structure of a patient, the model points being associated with a model space; collecting a set of measured points of the anatomic structure of the patient, the measured points being associated with a patient space; determining a first plurality of weights for the set of model points respectively based on a target anatomic location; and registering the set of model points to the set of measured points based on the first plurality of weights to generate a registration. In some embodiments, the determining the first plurality of weights includes: for each model point, determining a target distance between the model point and the target anatomic location; and assigning a weight to the measured point based on the target distance. In some embodiments, the determining the first plurality of weights includes: for each model point, determining a navigation path distance between the model point and a predetermined navigation path to the target anatomic location; and assigning the weight to the measured point based on at least the target distance and the navigation path distance. In some embodiments, the assigning the weight to the model point includes: determining that the target distance is greater than a predetermined target distance threshold; and assigning the weight having a value of zero to the model point. In some embodiments, a first model point has a first distance from the target anatomic location, wherein a second measured point has a second distance from the target anatomic location, the second distance being less than the first distance, and wherein a first weight assigned to the first model point is less than a second weight assigned to the second measured point.

One or more elements in embodiments of the invention may be implemented in software to execute on a processor of a computer system such as control system 112. When implemented in software, the elements of the embodiments of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device, The code segments may be downloaded via computer networks such as the Internet, Intranet, etc.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A system comprising:
a non-transitory memory;
one or more processors coupled to the non-transitory memory and configured to read instructions to cause the system to perform operations comprising:
accessing a set of model points of a model of an anatomic structure of a patient, the model points being associated with a model space;
collecting a set of measured points of the anatomic structure of the patient, the measured points being associated with a patient space;
determining a set of matches between the set of model points and the set of measured points;
determining a first plurality of weights for the set of matches based on a target anatomic location; and
registering the set of model points to the set of measured points based on the first plurality of weights to generate a first registration.

2. The system of claim 1, wherein the first plurality of weights for the set of matches is based on a proximity of each of the matches to an anatomic target, wherein the anatomic target is associated with the model space.

3. The system of claim 2, further comprising determining a second plurality of weights for the set of model points or the set of measured points.

4. The system of claim 3, wherein registering the set of model points to the set of model points is further based on the second plurality of weights.

5. The system of claim 1, further comprising obtaining a first distal end location of a distal end of a medical instrument inserted into the anatomic structure.

6. The system of claim 5, wherein the determining the first plurality of weights includes:
for each measured point, determining a distal end distance between the measured point and the first distal end location; and
assigning a weight to the measured point based on the distal end distance.

7. The system of claim 6, wherein the assigning the weight to the measured point based on the distal end distance includes:
determining that the distal end distance is greater than a predetermined distal end distance threshold; and
assigning the weight having a value of zero to the measured point.

8. The system of claim 6, wherein a first measured point has a first distance from the first distal end location,
wherein a second measured point has a second distance from the first distal end location, the second distance being less than the first distance, and
wherein a first weight assigned to the first measured point is less than a second weight assigned to the second measured point.

9. The system of claim 6, wherein the operations further comprise:
detecting a movement of the distal end to a second distal end location;
determining a second plurality of weights for the set of measured points respectively based on the second distal end location; and
registering the set of model points to the set of measured points based on the second plurality of weights to generate a second registration.

10. The system of claim 6, further comprising:
for each measured point, determining a target distance between the measured point and a target anatomic location; and
assigning the weight to the measured point based on at least one of the distal end distance and the target distance.

11. A system comprising:
a non-transitory memory;
one or more processors coupled to the non-transitory memory and configured to read instructions to cause the system to perform operations comprising:
accessing a set of model points of a model of an anatomic structure of a patient, the model points being associated with a model space;
collecting a set of measured points of the anatomic structure of the patient, the measured points being associated with a patient space;
determining a first plurality of weights for the set of model points respectively based on a target anatomic location; and
registering the set of model points to the set of measured points based on the first plurality of weights to generate a registration.

12. The system of claim 11, wherein the determining the first plurality of weights includes:
for each model point, determining a target distance between the model point and the target anatomic location; and
assigning a weight to the measured point based on the target distance.

13. The system of claim 12, wherein the determining the first plurality of weights includes:
for each model point, determining a navigation path distance between the model point and a predetermined navigation path to the target anatomic location; and
assigning the weight to the measured point based on at least the target distance and the navigation path distance.

14. The system of claim 12, wherein the assigning the weight to the model point includes:
determining that the target distance is greater than a predetermined target distance threshold; and
assigning the weight having a value of zero to the model point.

15. The system of claim 11, wherein a first model point has a first distance from the target anatomic location,
wherein a second measured point has a second distance from the target anatomic location, the second distance being less than the first distance, and
wherein a first weight assigned to the first model point is less than a second weight assigned to the second measured point.

16. A method performed by a computing system, the method comprising:
accessing a set of model points of a model of an anatomic structure of a patient, the model points being associated with a model space;
collecting a set of measured points of the anatomic structure of the patient, the measured points being associated with a patient space;
registering the set of model points with the set of measured points to generate a first registration;
dividing the anatomic structure into a plurality of anatomic areas;
generating a plurality of area registrations for the plurality of anatomic areas respectively based on the first registration; and
generating a second registration for translating the model space to the patient space using the plurality of area registrations.

17. The method of claim 16, wherein the generating the plurality of area registrations for the plurality of anatomic areas includes for each anatomic area:
determining a subset of measured points in the anatomic area based on the first registration; and
registering the model points in the anatomic area with the corresponding subset of measured points to generate a corresponding area registration.

18. The method of claim 16, wherein the dividing the anatomic structure into the plurality of anatomic areas includes:
dividing the anatomic structure based on structure rigidities of the plurality of anatomic areas respectively.

19.-20. (canceled)

21. The method of claim 16, wherein the generating the second registration includes:
blending the area registrations for adjacent anatomic areas in a transition area of the adjacent anatomic areas.

22. The method of claim 16, wherein the second registration includes at least one of a deflection estimate and a rotation estimate for a first anatomic area.
